(19) United States
(12) Patent Application Publication
(10) Pub. No.: US 2007/0232810 A1
(43) Pub. Date: Oct. 4, 2007

(54) Process for preparing retinoid compounds

(75) Inventor: Michael Martin, San Francisco, CA (US)

Correspondence Address: ROCHE PALO ALTO LLC;PATENT LAW DEPT. M/S A2-250, 3431 HILLVIEW AVENUE, PALO ALTO, CA, 94304, US

(73) Assignee: Roche Palo Alto LLC

(21) Appl. No.: 11/731,183

(22) Filed: Mar. 30, 2007

Related U.S. Application Data
(60) Provisional application No. 60/787,752, filed on Mar. 31, 2006.

(51) Int. Cl. C07D 231/12

(52) U.S. Cl. 548/377.1

(57) ABSTRACT

The application claims a process for the preparation of a RAR modulator according to formula Ia or Ib comprising to sequential Heck couplings steps to elaborated the disubstituted olefin.

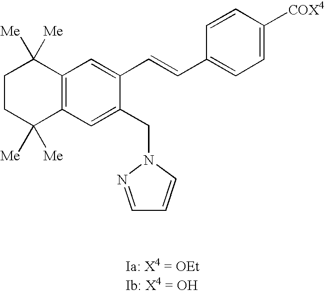

### CROSS REFERENCE TO PRIOR APPLICATIONS

This application claims the benefit of priority to U.S. Ser. No. 60/787,752 filed Mar. 31, 2006 the contents of which are hereby incorporated in their entirety by reference.

### FIELD OF THE INVENTION

The invention relates to an efficient and convenient palladium-catalyzed process for the preparation of novel retinoid compounds of formula Ia and/or Ib.

### BACKGROUND OF THE INVENTION

The retinoids are structural analogs of vitamin A and include both natural and synthetic compounds. Retinoid compounds such as all trans retinoic acid (“ATRA”), 9-cis-retinoic acid, trans 3,4-didehydroretinoic acid, 4-oxo retinoic acid, 13-cis-retinoic acid and retinol are pleiotrophic ligands that regulate a large number of inflammatory, immune and structural cells.

Retinoids modulate epithelial cell proliferation, morphogenesis in lung and differentiation through a series of hormone nuclear receptors that belong to the steroid/thyroid receptor superfamily. The retinoid receptors are classified as retinoic acid receptors (RAR) and retinoid X receptors (RXR) each of which consists of three distinct subtypes (α, β and γ). ATRA is the natural ligand for the retinoic acid receptors and binds with similar affinity to the α, β and γ subtypes. A number of synthetic RAR α, β and γ retinoid agonists have also been described in the art (See, e.g., Belloni et al., U.S. Pat. No. 5,962,508; Klaus et al., U.S. Pat. No. 5,986,131; J.-M. Lapierre et al., WO02/28810).

In tissues other than pulmonary tissues, retinoids typically have anti-inflammatory effects, can alter the progression of epithelial cell differentiation and may inhibit stromal cell matrix production. These biological effects of retinoids have led to the development of many topical agents for dermatological disorders such as psoriasis, acne, and hypertrophic cutaneous scars. Retinoids have also been used in the treatment of light and age damaged skin, the healing of wounds caused, for example, by surgery and burns (Mustoe et al., Science 237, 1333 1987; Sprugel et al., J. Pathol., 129, 601, 1987; Boyd, Am. J. Med., 86, 568, 1989) and as anti-inflammatory agents for treatment of arthritis. Other medicinal applications of retinoids include the control of acute promyelocytic leukemia, adeno and squamous cell carcinoma and hepatic fibrosis. Retinoids have also been used extensively in treatment of premalignant epithelial lesions and malignant tumors (carcinomas) of epithelial origin (Bollag et al., U.S. Pat. No. 5,248,071; Sporn et al., Fed. Proc. 1976, 1332; Hong et al., “Retinoids and Human Cancer” in The Retinoids: Biology, Chemistry and Medicine, M. B. Spom, A. B. Roberts and D. S. Goodman (eds.) Raven Press, New York, 1994, 597-630). A general review of retinoids can be found in Goodman & Gilman's “The Pharmacological Basis of Therapeutics”, 9^{th} edition (1996, McGraw-Hill) Chapters 63-64.

Chronic Obstructive Pulmonary Disease (“COPD”) refers to a large group of lung diseases including asthma, emphysema and chronic bronchitis which hinder normal respiration. Approximately 11% of the population of the United States has COPD and available data suggests that the incidence of COPD is increasing. Currently, COPD is the fourth leading cause of mortality in the United States. Severe manifestations of COPD include symptoms of emphysema particularly chronic shortness of breath. No effective methods for reversing the clinical indications of emphysema currently exist in the art. ATRA has been reported to induce formation of new alveoli and returns elastic recoil in the lung to approximately normal values in animal models of emphysema (Massaro et al., Nature Med., 1997, 3, 675; “Strategies to Augment Alveolization,” National Heart, Lung, and Blood Institute, RFA: HL-98-011, 1998; Massaro et al., U.S. Pat. No. 5,998,486).

Thus retinoids have significant potential in treating serious health problems. Compound I and related compounds have been shown to be specific RARγ agonists which are useful in treating the above mentioned diseases. Thus an efficient process for the preparation of Ia or Ib is desirable.

Synthetic routes to retinoid compounds have been reviewed. (B. Dominguez et al. Org. Prep. Proceed. Int. 2003 35(3):239-306; M. I. Dawson and P. D. Hobbs, In “The Synthetic Chemistry of Retinoids”, M. R. Spoon, A. B. Roberts and D. S. Goodman (Eds.), The Retinoids: Biology, Chemistry and Medicine, 2^{nd} edition, Raven, N.Y. 1994, p. 5; R. S. H. Liu and A. Asato, Tetrahedron 1984 40:1931). Retinoid receptor modulators comprise a structurally diverse group of compounds; however, olefins, polyolefins, bis-aryl and acetylenic linkages constitute common structural motifs. Accordingly, established olefination reactions utilizing phosphonium ylides (Wittig reaction), phosphonium salts (Horner-Wadsworth-Emmons reaction), sulfone coupling (Julia olefin synthesis) are commonly used in retinoid synthesis. Recently, transitional metal catalyzed Cₛₚ₂-Cₛₚ₂ and Cₛₚ₂-Cₛₚ cross coupling reactions have been applied to the synthesis of retinoid modulators. Variants of metal catalyzed cross-couplings include the Negishi coupling, the Stille reaction, the Suzuki reaction and the Heck reaction.

1,2-bis-Aryl-ethene compounds have useful properties as selective RAR agonists. Among this group of RAR agonists, 4-[(E)-2-(5,5,8,8-tetramethyl-3-pyrazol-1-ylmethyl-5,6,7,8-tetrahydro-naphthalen-2-yl)-vinyl]-benzoic acid (Ib) is a particularly useful RARγ selective agonist. A synthesis of 4-[(E)-2-(5,5,8,8-tetramethyl-3-pyrazol-1-ylmethyl-5,6,7,8-tetrahydro-naphthalen-2-yl)-vinyl]-benzoic acid has been disclosed by J.-M. Lapierre et al. in WO02/28810 published Apr. 11, 2002 which utilizes a phosphonate coupling to introduce the E-olefin (SCHEME A).

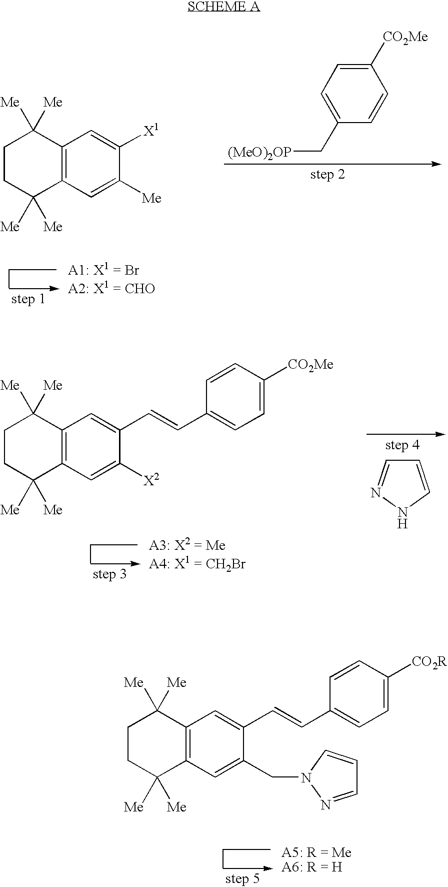

One variant of palladium-mediated couplings, the Suzuki reaction, has been employed to elaborate the E-disubstituted olefin in the preparation of 4-[2-(5,5,8,8-tetramethyl-5,6,7,8-tetrahydro-naphthalen-2-yl)-vinyl]-benzoic acid (A. Torrado et al., Synthesis 1995 285; SCHEME B). The Suzuki reaction, however, requires the multi-step sequence including formation of a boronic acid prior to the palladium-catalyzed coupling step.

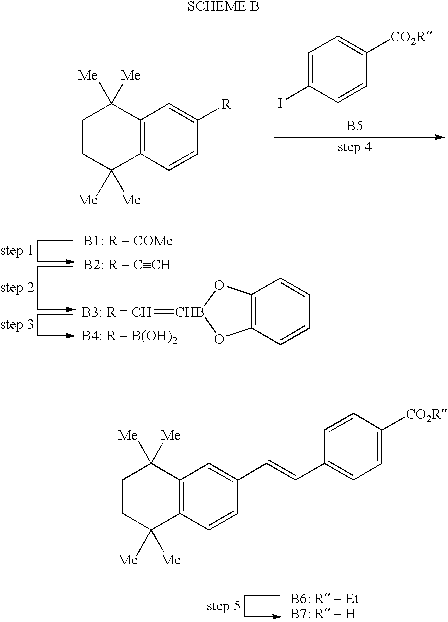

The Heck reaction (vide infra) has been utilized to introduce a vinyl substitutent onto an aromatic ring. M. Reetz et al. (Angew. Chem. Int. Ed. Eng. 1998 37(4):481-483; WO98/42664) has disclosed the efficient olefination of 6-methoxy-2-bromo-naphthalene (C3) to afford C4. S. Gibson et al. (Chem. Commun. 2001 779-780) have reported phosphapalladacyclic complexes which catalyze the same transformation. LaPierre et al. (supra) disclose the olefination of C1 with trimethoxysilylethane to afford the vinyl naphthalene C2.

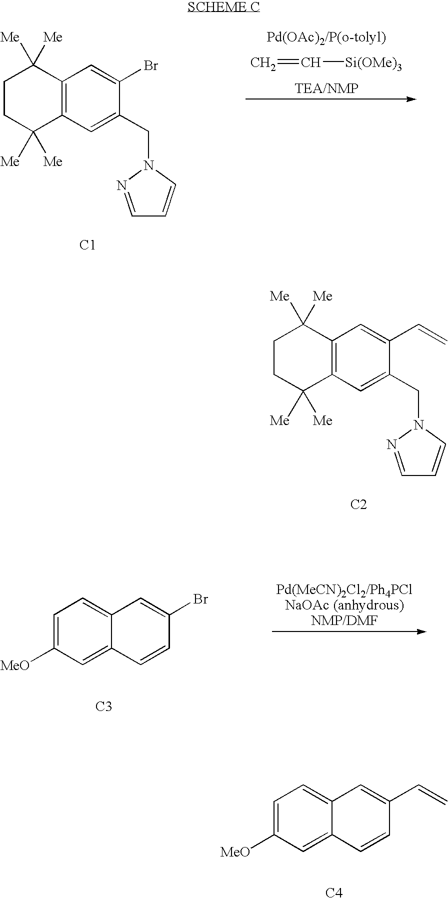

The process herein claimed exploits two sequential Heck arylations to form an unsymmetrical ethene compound which are carried out in a single reaction vessel without isolation of the intermediate styrene derivative. The formation of stilbenes from aryl halides and ethene has been disclosed (J. E. Plevyak and R. F. Heck, J. Org Chem. 1978 43(12):2454-2456). Coupling of two biphenyl halides with ethylene to afford symmetrical stilbene dyes has been described (J. Rümper et al., Chemische Berichte/Recueil 1997 130(9):1193-1195). These documents describe symmetrical coupling or aryl halides and ethylene resulting in a symmetrical stilbene. In contrast, the present invention describes a process allowing incorporation of dissimilar substituents into an ethylene moiety.

### SUMMARY OF THE INVENTION

The present invention relates to a process for preparing a compound of formula Ia comprising two sequential Heck olefin couplings which can be carried in one vessel without isolation an intermediate and which optionally includes the hydrolysis of Ia to Ib comprising the steps of:

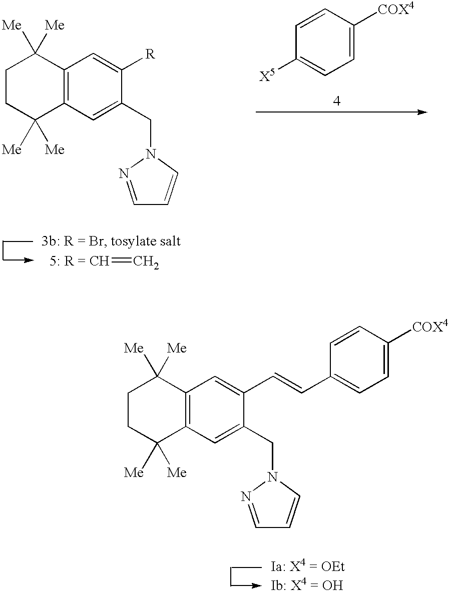

(i) exposing a solution of 3b, a first base, a palladium compound and optionally a phosphine ligand in a polar organic solvent to ethylene at a temperature and pressure sufficient to initiate the replacement of bromine substituent with ethylene and afford 5;
(ii) contacting the resulting solution containing 5 with 4-substituted benzoic acid derivative 4 wherein X⁴ is OH or C₁₋₆ alkoxy, X⁵ is a leaving group susceptible to palladium catalyzed displacement, optionally adding, independently of each other, additional base and/or palladium compound and/or phosphine ligand, at a temperature sufficient to initiate the replacement of bromine substituent with 5 and afford Ia;
(iii) optionally contacting Ia with a hydroxide source in an organic solvent optionally containing water, and isolating the crystalline carboxylic acid Ib.

### DETAILED DESCRIPTION OF THE INVENTION

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

“Optional” or “optionally” means that a subsequently described event or circumstance may but need not occur, and that the description includes instances where the event or circumstance occurs and instances in which it does not. For example, “optional bond” means that the bond may or may not be present, and that the description includes single, double, or triple bonds.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

An efficient process for the preparation of Ia or Ib and related analogs has been identified which comprises two sequential Heck coupling reactions which can be carried out without isolation of intermediates to produce non-symmetrical olefins of formula Ia or Ib. The process has the added advantage that compounds which are potent RAR receptors modulators are not formed until the final step in the reaction sequence which limits worker exposure to potent pharmacologically active compounds.

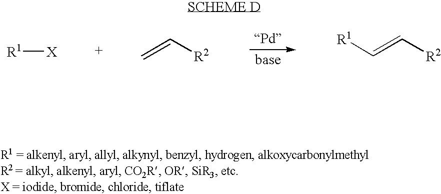

The Heck reaction is broadly defined as the coupling of alkenyl or aryl (sp²) halides or triflates with alkenes which formally results in the replacement of a hydrogen atom in the alkene coupling partner by R¹. The Heck reaction has been reviewed. (R. F. Heck in Organic Reactions, vol. 24 Malabar 1984; G. T. Crisp, Chem. Soc. Rev. 1998 27:427; S. Bräse and S. de Meijere, “Palladium-catalysed Coupling of Organic Halides to Alkenes—The Heck Reaction” in Metal-Catalyzed Cross Coupling Reactions, F. Diederich and P. J. Stang, Eds. Wiley-VCH, Weiheim 1997 p. 99-166).

The Heck reaction is catalyzed by palladium. The valence state of the active catalytic species is assumed to be Pd(0) which can be produced in situ by reduction of a Pd(II) species with phosphine ligands. Palladium compounds reported to be useful in the Heck reaction include, but are not limited to, Pd(II)(OAc)₂, Pd(PPh₃)₄, PdCl₂(PPh₃)₂, Pd(MeCN)₂Cl₂, Pd(acac)₂, Pd(dba)₂, Pd₂(dba)₃ and Pd on solid supports. While the present process is exemplified with Pd(II)(OAc)₂, one skilled in the art will appreciate that other Pd catalysts could be substituted with departing from the spirit of the invention. The term “palladium compound” as used herein refers to a palladium compound capable of producing a catalytically active species that catalyzes the replacement of a bromo-, iodo- or trifluorosulfonyloxy radical with an olefin. The catalytically active palladium species generally have been phosphine ligands. Triphenylphosphine is used commonly. The term “phosphine ligand” as used herein refers to triaryl and triheteroarylphosphines. The term also refers to bidentate ligands which have proven to be effective catalysts including, but not limited to, 1,2-bis-(diphenylphosphino)ethane (dppe), 1,3-bis-(diphenylphosphino)propane (dppp), 1,2-bis-(diphenylphosphino)butane (dppb) and 1,1′-bis-(diphenylphosphino)ferrocene (pddf). The term “phosphine ligand” also encompasses trialkylphosphines such as tri(tert-butyl)phosphine and the like which have be used advantageously in palladium-catalyzed coupling reactions. The palladium compound used herein may contain coordinated phosphine ligands or a palladium compound such as Pd(OAc)₂ may be used and phosphine ligands added separately. Either route is within the contemplated scope of the invention.

The utility of the Heck reaction in organic synthesis has stimulated extensive research to identify bases, ligands, additives and reaction conditions which will enhance reactivity and regioselectivity of the reaction. Bases which have be reported include, but are not limited to, TEA, ethylenediamine, DABCO and other secondary and tertiary amines, K₂CO₃, Na₂CO₃, KO^{t}Bu, NaOAc, K₂CO₃, CaCO₃ have been incorporated into the reaction mixture. Alkali metal hydroxides are commonly included in the reaction medium. The phrase “first base” as used herein refers to an organic or inorganic base which promotes palladium-catalyzed coupling including, but not limited to, the aforementioned list. Silver (I) and thallium (I) salts have used as additives in the Heck reaction. Phase transfer conditions have also been advantageously adapted to the Heck Reaction (T. Jeffery Tetrahedron Lett. 1985 26:2667-2670)

A wide range of solvents can be used to carry out the Heck reaction including DMF, DMA, NMP, MeCN, DMSO, MeOH, EtOH, tert-butanol, THF, dioxane, benzene, toluene, mesitylene, xylene, CHCl₃ and DCE. The reaction is most commonly run in polar aprotic solvents such as the first five exemplified above. It is apparent however that a wide range of solvents are compatible with the Heck reaction and a determinative feature often is solubility and the temperature required to effect the transformation. The phrase “polar organic solvent” as used herein refers to DMF, NMP, DMSO, DMA and MeCN.

The term “organic solvent” refers to the solvent or solvents used in the hydrolysis of the carboxylic ester. One skilled in the art will recognized that many solvents can be used including water miscible and water immiscible solvents. The choice of the first solvent is primarily a matter of operational convenience and useful examples include, but are not limited to, lower alcohols and aqueous lower alcohols. In one embodiment of the invention the first solvent is a solution of ethanol and water.

The phrase “first non-polar organic solvent” refers to a solvent suitable for the free radical bromination of the benzyl substituent. Acceptable solvents generally include halocarbons and hydrocarbons; however, one skilled in the art could readily confirm the suitability of other specific solvents which are inert under the reaction conditions and also are within the scope of the invention. Commonly used solvents include cyclohexane, carbon tetrachloride and CF₃—C₆H₅.

The phrase “free radical brominating agent” as used herein refers to a reagent capable of producing bromine free radicals under the reaction conditions. Typical reagents which can be used as a source for bromine free radicals include bromine, N-bromo-succinimide and 1,3-dibromo-5,5-dimethyl-imidazolidine-2,4-dione. The phrase “free radical initiator” as used herein refers to reagents which can generate bromine free radicals under reaction conditions. Commonly free radical initiators include AIBN and 2,2′-azo-bis-(2,4-dimethyl-pentanenitrile) (Vazo® 52). Light can also be used to initiate the formation of bromine radicals and accordingly is within the scope of the invention.

The phrase “electrophilic brominating reagent” as used herein refers to a reagent which generates an electropositive bromine atom capable of brominating an aromatic ring. Bromine in the presence of Lewis acids or protic acids is commonly as an electrophilic brominating reagent. The combination of HBr and H₂O₂ results in the in situ production of Br₂. Other sources of electropostive bromine are well known in the art and are with in scope of the invention.

The phrase “4-substituted benzoic acid derivative” as used herein refers to a benzene ring substituted with a leaving group para to the carboxylic acid or ester which can be replaced by ethylene under the coupling conditions and which generally include halogen or trifluorosulfonyloxy. The ester or acid can be replaced by any group which is compatible with the reaction conditions and which can be readily transformed into a carboxylic acid or ester.

The term “alkyl” as used herein denotes an unbranched or branched chain, saturated, monovalent hydrocarbon residue containing 1 to 10 carbon atoms. The term “lower alkyl” denotes a straight or branched chain hydrocarbon residue containing I to 6 carbon atoms. “C₁-₁₀ alkyl” as used herein refers to an alkyl composed of 1 to 10 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁-₁₀ alkoxy” as used herein refers to an —O-alkyl wherein alkyl is C₁-₁₀. The term Lower alcohol refers means an R—OH compound wherein R is lower alkyl as herein defined.

The term “carboxylic acid” as used herein refers to a compound R—C(═O)OH were R is an alkyl group as herein defined.

The term “second base” as used herein refers to a base which is used to trap HBr formed by the displacement of the benzylic bromide with pyrazole. Many bases are used for this purposed including amine bases such as alkali metal phosphates (including mono-, di- and tri-alkali metal phosphates), TEA, DABCO, DIPEA and pyridine, alkali or alkaline metal carbonates and hydrogen carbonate and alkaline or alkali metal carboxylates and all variants are within the scope of the invention.

In one embodiment of the present invention there is provided a process for preparing a compound of formula Ia or Ib employing sequential Heck reactions comprising the steps of: (i) exposing a solution of aryl bromide 3b, optionally as a acid addition salt, a first base, a palladium compound and optionally a phosphine ligand in a polar organic solvent to ethylene at a temperature and pressure sufficient to initiate the replacement of bromine substituent with ethylene and to afford 5 in a first Heck reaction and subsequently (ii) contacting the resulting solution containing 5 with a 4-substituted benzoic acid derivative 4 wherein X⁴ is OH or C₁₋₆ alkoxy and X⁵ is a leaving group susceptible to palladium catalyzed displacement. In the present embodiment the ester is optionally hydrolyzed to the corresponding carboxylic acid Ib by contacting Ia with a hydroxide source in an organic solvent optionally containing water to afford the crystalline carboxylic acid Ib. While it is advantageous to use the solution of 5 obtained from the first Heck reaction directly in the second Heck reaction, one skilled in the art would clearly recognize that the styrene intermediate could be isolated without departing from the spirit of the invention.

In this and the other embodiments, additional base and/or palladium compound and/or phosphine ligand can be added prior to the second Heck reaction to realize satisfactory reaction rates. Typically the added reagents are the same as those used initially, however, alternative reagents that fall within the scope of the invention could be added. Similarly the temperature and ethylene pressure can be adjusted to initiate and maintain the replacement of bromine substituent with 5 to afford Ia in a second Heck reaction. In all embodiments the initially formed ester, e.g., Ia is optionally hydrolyzed to the corresponding carboxylic acid by contacting the ester with a hydroxide source in an organic solvent optionally containing water. Hydrolysis of esters is a routine transformation in organic synthesis and many alternative conditions exist which are within the scope of the invention.

In another embodiment of the present invention there is provided a process for preparing a compound of formula Ia or Ib comprising the steps of: (i) exposing a solution of a salt of aryl bromide 3b, a tertiary amine, Pd(II)(OAc)₂ and tris-(o-tolyl)phosphine in a polar organic solvent to ethylene at a temperature and pressure sufficient to initiate the replacement of bromine substituent with ethylene and afford styrene 5 in a first Heck reaction and subsequently (ii) contacting the resulting solution containing 5 with a 4-substituted benzoic acid derivative 4 wherein X⁴ is an lower alkoxy and X⁵ is bromo, iodo or trifluorosulfonyloxy. Additional tertiary amine, Pd(II)(OAc)₂ and tris-(o-tolyl)phosphine are optionally added prior to the second Heck reaction and the temperature and ethylene pressure is maintained at a level sufficient to initiate the replacement of bromine or iodo substituent with 5 to afford stilbene Ia. In this embodiment the ester is optionally hydrolyzed to the corresponding carboxylic acid Ib by contacting Ia with a hydroxide source in a lower alcohol and/or ether solvent optionally containing water to afford the crystalline carboxylic acid Ib.

In a related embodiment the esterified 4-substituted benzoic acid derivative in the second Heck reaction is an ester of 4-bromobenzoic acid.

In another embodiment of the present invention there is provided a process for preparing a compound of formula Ia or Ib comprising the steps of: (i) exposing a solution of the tosylate salt of 3b, Pd(II)(OAc)₂, tris-(o-tolyl)phosphine and TEA in NMP to ethylene at a temperature and pressure sufficient to initiate the replacement of bromine substituent with ethylene and afford 5 and subsequently (ii) contacting the resulting solution containing S with ethyl p-bromo-benzoate. Additional TEA, or an equivalent base, Pd(II)(OAc)₂ and tris-(o-tolyl)phosphine can optionally be added prior to the second Heck reaction and the temperature is maintained at a level sufficient to initiate the replacement of the bromine substituent with 5 to afford Ia. The ester is optionally hydrolyzed to the corresponding carboxylic acid Ib by contacting Ia with NaOH in an aqueous EtOH to afford the crystalline carboxylic acid Ib.

In another embodiment of the present invention there is provided a process for preparing a compound of formula Ia or Ib employing two sequential Heck reactions comprising the steps (i) contacting a solution of 2b in a non-polar organic solvent with a free radical brominating reagent and a free radical initiator at a temperature sufficient to initiate the bromination of benzylic methyl substituent to afford a solution of 3a, (ii) contacting said solution of 3a with pyrazole and optionally a second base capable of trapping hydrogen bromide, (iii) partitioning the resulting solution between water and toluene and isolating 3b as an acid addition salt or a free base and (iv) exposing a solution of 3b, optionally as a acid addition salt, a first base, a palladium compound and optionally a phosphine ligand in a polar organic solvent to ethylene at a temperature and pressure sufficient to initiate the replacement of bromine substituent with ethylene and afford 5 in a first Heck reaction and subsequently (v) contacting the resulting solution containing 5 with a 4-substituted benzoic acid derivative 4 wherein X⁴ is lower alkoxy and X⁵ is a leaving group susceptible to palladium catalyzed displacement. Additional base and/or palladium compound and/or phosphine ligand can be added prior to the second Heck reaction and the temperature is maintained at a level sufficient to initiate the replacement of bromine substituent with 5 to afford Ia in a second Heck reaction. The ester is optionally hydrolyzed to the corresponding carboxylic acid Ib by contacting Ia with a hydroxide source in a lower alcohol and/or ether solvent, optionally containing water, to afford the crystalline carboxylic acid Ib. While it is advantageous to use the solution of 5 obtained from the first Heck reaction directly in the second Heck reaction, the styrene could be isolated without departing from the spirit of the invention.

In another embodiment of the present invention there is provided a process for preparing a compound of formula Ia or Ib employing two sequential Heck reactions comprising the steps (i) contacting a solution of 2b in cyclohexane with 1,3-dibromo-5,5-dimethyl-imidazolidine-2,4-dione and 2,2′-azo-bis-(2,4-dimethyl-pentanenitrile) at a temperature sufficient to initiate the bromination of benzylic methyl substituent to afford a solution of 3a, (ii) contacting said solution of 3a with pyrazole and tribasic potassium phosphate, (iii) partitioning the resulting solution between water and toluene and isolating 3b as an acid addition salt or as a free base, (iv) exposing a solution of 3b, TEA, Pd(II)(OAc)₂ and tris(o-tolyl)phosphine in NMP to ethylene at a temperature and pressure sufficient to initiate the replacement of bromine substituent with ethylene and afford 5 in a first Heck reaction and subsequently (v) contacting the resulting solution containing 5 with ethyl p-bromo-benzoic acid. Additional TEA, Pd(II)(OAC)₂ and tris(o-tolyl)phosphine can be added prior to the second Heck reaction and the temperature and ethylene pressure is maintained at a level sufficient to initiate the replacement of bromine substituent with 5 to afford stilbene Ia in a second Heck reaction. In the present embodiment the ester is optionally hydrolyzed to the corresponding carboxylic acid Ib by contacting Ia with NaOH in aqueous EtOH to afford the crystalline carboxylic acid Ib.

In another embodiment of the present invention there is provided a process for preparing a compound of formula Ia or Ib employing two sequential Heck reactions comprising the steps (i) contacting a solution of 2,5-dimethyl-2,5-dihydroxy-hexane (1a) and toluene with aqueous HCl and isolating 2,5-dimethyl-2,5-dichloro-hexane (1b), (ii) contacting a solution of 1b and toluene with a Lewis acid and isolating 2a, (iii) contacting a solution of 2a and a carboxylic acid with an electrophilic brominating reagent to afford 2b which is optionally isolated, (iv) contacting a solution of 2b and a non-polar organic solvent with a free radical brominating reagent and a free radical initiator at a temperature sufficient to initiate the bromination of benzylic methyl substituent to afford a solution of 3a, (v) contacting said solution of 3a with pyrazole and optionally a second base capable of trapping HBr, (vi) partitioning the resulting solution between water and toluene and isolating 3b as an acid addition salt or a free base, (vii) exposing a solution of 3b, optionally as a acid addition salt, a first base, a palladium compound and optionally a phosphine ligand in a polar organic solvent to ethylene at a temperature and pressure sufficient to initiate the replacement of bromine substituent with ethylene and afford 5 in a first Heck reaction and subsequently (viii) contacting the resulting solution containing 5 with a 4-substituted benzoic acid derivative 4 wherein X⁴ is lower alkoxy and X⁵ is a leaving group susceptible to palladium catalyzed displacement.

In another embodiment of the present invention there is provided a process for preparing a compound of formula Ia or Ib employing two sequential Heck reactions comprising the steps (i) contacting a solution of 2,5-dimethyl-2,5-dihydroxy-hexane (1a) in toluene with aqueous hydrochloric acid and isolating 2,5-dimethyl-2,5-dichloro-hexane (1b), (ii) contacting a solution of 1b in a toluene with AlCl₃ and isolating 2a, (iii) contacting a solution of 2a with bromine to afford 2b which is optionally isolated, (iv) contacting a solution of 2b in cyclohexane with a 1,3-dibromo-5,5-dimethyl-imidazolidine-2,4-dione and 2,2′-azo-bis-(2,4-dimethyl-pentanenitrile) at a temperature sufficient to initiate the bromination of benzylic methyl substituent to afford a solution of 3a, (v) contacting said solution of 3a with pyrazole and tribasic potassium phosphate (vi) partitioning the resulting solution between water and toluene and isolating 3b as an acid addition salt or a free base, (vii) exposing a solution of 3b, optionally as an acid addition salt, TEA, Pd(II)(OAc)₂ and tris-(o-tolyl)phosphine in a NMP to ethylene at a temperature and ethylene pressure sufficient to initiate the replacement of bromine substituent with ethylene and to afford 5 in a first Heck reaction and subsequently (viii) contacting the resulting solution containing 5 with an ethyl p-bromo-benzoate. Additional TEA, Pd(II)(OAc)₂ and tris-(o-tolyl)phosphine can be optionally added prior to the second Heck reaction and the temperature is maintained at a level sufficient to initiate the replacement of bromine substituent with 5 to afford Ia in a second Heck reaction. In the present embodiment the ester is optionally hydrolyzed to the corresponding carboxylic acid Ib by contacting Ia with NaOH and aqueous EtOH to afford the crystalline carboxylic acid Ib.

Commonly used abbreviations include: acetyl (Ac), azo-bis-isobutyrylnitrile (AIBN), atmospheres (Atm), benzyl (Bn), butyl (Bu), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), 1,2-dichloroethane (DCE), dichloromethane (DCM), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), (diphenylphosphino)ethane (dppe), (diphenylphosphino)ferrocene (dppf), ethyl (Et), ethyl acetate (EtOAc), ethanol (EtOH), diethyl ether (Et₂O), acetic acid (HOAc), high pressure liquid chromatography (HPLC), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-chlorosuccinimide (NCS), N-methylmorpholine (NMM), N-methylpyrrolidone (NMP), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), triflate or CF₃SO₂— (Tf), trifluoroacetic acid (TFA), 1,1′-bis-2,2,6,6-tetramethylheptane-2,6-dione (TMHD), thin layer chromatography (TLC), tetrahydrofuran (THF), trimethylsilyl or Me₃Si (TMS), p-toluenesulfonic acid monohydrate (TsOH or pTsOH), 4-Me—C₆H₄SO₂— or tosyl (Ts). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

The following example illustrates the process disclosed herein. This example is provided to enable those skilled in the art to more clearly understand and to practice the present invention and it should not be considered as limiting the scope of the invention, but merely as being illustrative and representative thereof.

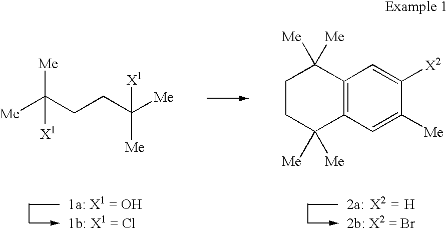

step A—A 100 gal glass-lined reactor was charged with 1a (CAS Reg. No. 110-03-2, 18.1 Kg), toluene (30.4 Kg) and 37% HCl (225 Kg). The biphasic mixture was stirred overnight at RT. After draining off the lower layer, the toluene solution was added to AlCl₃ (1.23 Kg) over 1 h. The mixture was aged at 60° C. for 2 h. An aqueous HCl solution (2.93 Kg of 37% HCl diluted with 7.07 Kg water) was added to the reaction mixture and the lower layer discarded. The organic layer was washed with additional water (5.07 L), the phases separated and the toluene was removed by vacuum distillation and replaced with propionic acid (20.42 Kg). The solution was further concentrated until less than 0.1 % toluene remains. Additional propionic acid (6.17 Kg), H₂O (12.3 L) and 48% HBr (19.9 Kg) were added followed by 30% H₂O₂ added over 1 h while maintaining the internal temperature between 50-60° C. The reaction was stirred for 1 h a the addition was complete then the temperature was raised to 80° C. for one additional hour. The reaction was quenched with sodium sulfite solution (2 Kg Na₂SO₃ and 19.3 Kg of H₂O) followed by an additional H₂O (72 L). The resulting mixture was aged overnight at 20° C. then filtered to give to afford 28.6 Kg (82% yield for the 3 steps) of 2b.

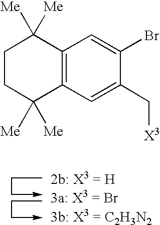

step B—A 100 gallon glass-lined reactor was charged with 2b (28 Kg), 1,3-dibromo-5,5-dimethyl-imidazolidine-2,4-dione (22.83 Kg), 2,2′-azo-bis-(2,4-dimethyl-pentanenitrile) (Vazo® 52, E. I. DuPont de Nemours) (481 g) and cyclohexane (151 Kg). The mixture was warmed to 55° C. resulting in a slow exotherm with the temperature rising to 65° C. After 1 h at 65° C. the reaction was quenched with aqueous sodium sulfite solution (19.3 Kg Na₂SO₃ in 166 L of H₂O). The aqueous layer was drained off and the organic layer washed with water (100 L). The cyclohexane was removed by distillation at atmospheric pressure and replaced with NMP (75 Kg). The resulting solution was transferred to a dry mixture of pyrazole (6.83 Kg) and K₃PO₄ (21 Kg). Additional NMP (18 Kg) was added and the mixture was heated to between 105 and 120° C. for 2.5 h. The solution was transferred into 150 L of water. Toluene (98 Kg) and additional water (26 L) were added and the layers were separated after aging overnight. The aqueous layer was back extracted with toluene (52 Kg) and the combined toluene fractions were washed twice with water (100 L). The resulting toluene solution was added to p-TsOH.H₂O (16 Kg). Additional toluene (33 Kg) was added and the mixture was heated to 58° C. until the solution was homogenous. The mixture was cooled to around 45° C. where crystals first appear and then to 10° C. The resulting slurry was filtered and washed with additional toluene (39 Kg). The crude product (37 Kg) was suspended in toluene (250 Kg), aqueous NaOH (9 Kg 50% NaOH and 46 Kg of H₂O) was added and the mixture was heated to 40° C. The aqueous layer was removed and the toluene solution was washed with H₂O (50 L). The resulting toluene solution was added to carbon (2 Kg) and aged for several hours before filtering through a CELITE® pad (5 Kg). The cake is washed with additional toluene (30 Kg) and the combined toluene filtrates were added a solution of p-TsOH.H₂O in MeOH (13.57 Kg p-TsOH.H₂O and 25 Kg MeOH). About 50 Kg of the MeOH was removed by distillation and the remaining solution was cooled slowly to 5° C. and aged overnight. Filtration, washing with toluene (50 Kg) and vacuum drying (vacuum oven 50° C. with nitrogen bleed) afforded 24 Kg (46% overall) of the tosylate salt of 3b.

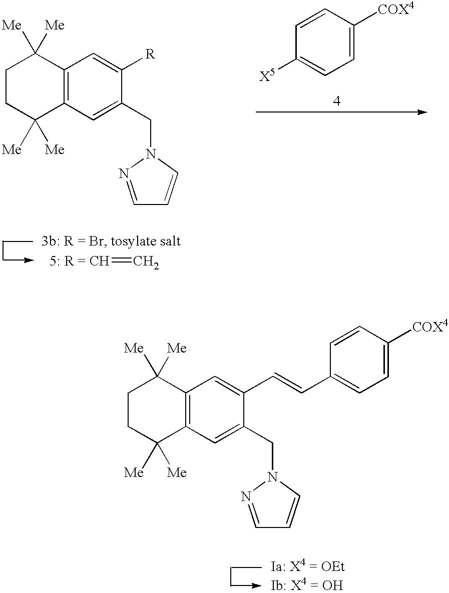

step C—A stock solution of the tosylate salt of 3b (24 Kg), TEA (16 Kg), Pd(OAc)₂ (24.1 g), tri-o-tolylphosphine (72 g) and NMP (73 Kg) was prepared and degassed with 3 vacuum/nitrogen cycles. This stock solution is stable if protected from heat and oxygen for at least 1 week. In six successive runs one sixth of the stock solution was introduced into a pressure (300 psi bursting disk) reactor and ethylene was introduced to 150 psi. The temperature was raised to 120° C. while the internal pressure is raised to 200 psi by additional ethylene. After 3 h the temperature was lowered to 80° C. and ethylene was vented. The resulting slurry was transferred to a holding vessel. After all six runs are completed, the combined slurries were charged with additional Pd(OAc)₂ (24 g), tri-o-tolylphosphine (72 g), and TEA (5.6 Kg). To the mixture is added ethyl p-bromo-benzoate and the temperature was raised to 105° C. The reaction was stirred for 5 h, cooled and partitioned between cyclohexane (150 Kg) and water (70 L). The water layer was withdrawn and the cyclohexane solution washed twice with water (2×60 L). After most of the cyclohexane was removed by atmospheric distillation, water (120 L) was added and the remaining cyclohexane removed by distillation. To the residue was added ethanol (140 Kg), H₂O (35 L) and 50% NaOH (24 Kg) and the mixture heated at reflux for 14 h. The temperature was lowered to 60° C. and the mixture filtered through a CELITE® pad (3 Kg). The cake was washed twice with a 1:1 (v:v) water ethanol mixture (2×26 Kg). The bulk of the ethanol was removed by distillation at atmospheric pressure. H₂SO₄ was introduced (20 Kg) followed by THF (178 Kg) and the layers were allowed to separate after thorough mixing. The lower aqueous layer was drained and the THF solution was clarified by filtration. The THF was removed by distillation at atmospheric pressure and replaced with n-butyl acetate (140 Kg total) at a rate that the volume remained relatively constant. The acid Ib crystallized during the solvent replacement. The temperature was reduced to 10° C. and the mixture aged overnight. The material was filtered and washed with n-butyl acetate (21 Kg) to afford 15 Kg (78%) of Ib.

The features disclosed in the foregoing description, or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A process for preparing a compound of formula Ia or Ib comprising the steps of: 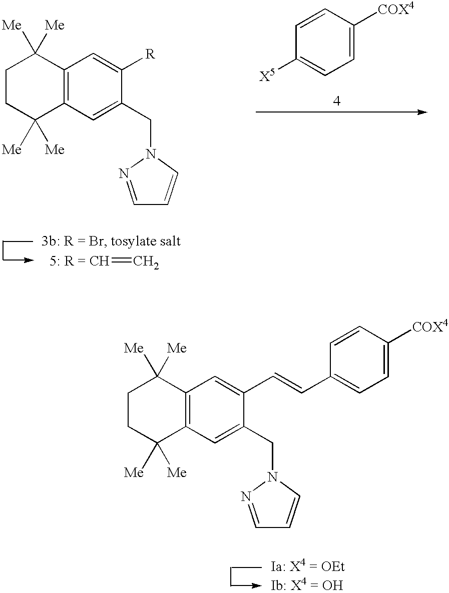
(i) exposing a solution of 3b, a first base, a palladium compound and optionally a phosphine ligand in a polar organic solvent to ethylene at a temperature and pressure sufficient to initiate the replacement of bromine substituent with ethylene and afford 5;
(ii) contacting the resulting solution containing 5 with 4-substituted benzoic acid derivative 4 wherein X⁴ is OH or C₁₋₆ alkoxy, X⁵ is a leaving group susceptible to palladium catalyzed displacement, optionally adding independently of each other additional base and/or palladium compound and/or phosphine ligand, at a temperature sufficient to initiate the replacement of bromine substituent with 5 and afford Ia;
(iii) optionally contacting Ia with a hydroxide source in an organic solvent optionally containing water, and isolating the crystalline carboxylic acid Ib.

2. A process according to claim 1 wherein said first base is a tertiary amine, said palladium compound is Pd(II)(OAc)₂, said phosphine ligand is tris-(o-tolyl)phosphine, said organic solvent is a lower alcohol and/or an ether and said 4-substituted benzoic acid derivative is an alkyl p-bromo-benzoate, alkyl p-iodo-benzoate or alkyl p-trifluoromethanesulfonyloxy-benzoate.

3. A process according to claim 2 wherein said first base is triethylamine, said 4-substituted benzoic acid derivative is ethyl p-bromo-benzoate, said organic solvent is aqueous ethanol and said polar organic solvent is N-methyl-pyrrolidone.

4. A process according to claim 1 said process further comprising the steps of: 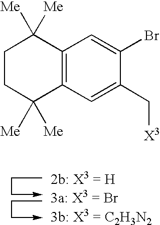
(i) contacting a solution of 2b in a non-polar organic solvent with a free radical brominating reagent, a free radical initiator, at a temperature sufficient to initiate the bromination of benzylic methyl substituent to afford a solution of 3a,
(ii) contacting said solution of 3a with pyrazole and optionally a second base capable of scavenging hydrogen bromide,
(iii) partitioning the resulting solution between water and toluene and isolating 3b as an acid addition salt or a free base.

5. A process according to claim 4 wherein said first base is a triethylamine, said palladium compound is Pd(II)(OAc)₂, said phosphine ligand is tris-(o-tolyl)phosphine, said organic solvent is aqueous ethanol, said 4-substituted benzoic acid derivative is ethyl p-bromo-benzoate, said polar organic solvent is N-methyl-pyrrolidone, said non-polar organic solvent is cyclohexane, said free radical brominating reagent is 1,3-dibromo-5,5-dimethyl-imidazolidine-2,4-dione, said free radical initiator is 2,2′-azo-bis-(2,4-dimethyl-pentanenitrile) and said second base is tribasic potassium phosphate.

6. A process according to claim 4 said process further comprising the steps of 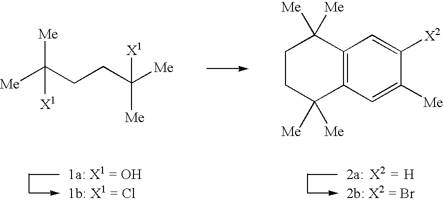
(i) contacting a solution of 2,5-dimethyl-2,5-dihydroxy-hexane (1a) and toluene with aqueous hydrochloric acid and isolating 2,5-dimethyl-2,5-dichloro-hexane (1b);
(ii) contacting a solution of 1b and toluene with a Lewis acid and isolating 2a; and,
(iii) contacting a solution of 2a and a carboxylic acid with an electrophilic brominating reagent to afford 2b which is optionally isolated.

7. A process according to claim 6 wherein said first base is a triethylamine, said palladium compound is Pd(II)(OAc)₂, said phosphine ligand is tris-(o-tolyl)phosphine, said first organic solvent is a lower alcohol and THF, said 4-substituted benzoic acid derivative is ethyl p-bromo-benzoate, said polar organic solvent is N-methyl-pyrrolidone, said first non-polar organic solvent is cyclohexane, said free radical brominating agent is 1,3-dibromo-5,5-dimethyl-imidazolidine-2,4-dione, said second base is tribasic potassium phosphate, said Lewis acid is AlCl₃, said carboxylic acid is propionic acid and said electrophilic brominating agent is hydrogen peroxide and hydrogen bromide.
